(19) United States
(12) Patent Application Publication
(10) Pub. No.: US 2010/0292283 A1
(43) Pub. Date: Nov. 18, 2010

(54) NOVEL PHENYL-ACETAMIDE AND PHENYL-PROPIONAMIDE DERIVATIVES USEFUL AS POTASSIUM CHANNEL MODULATORS

(76) Inventors: Antonio Nardi, Herzogenrath (DE); Jeppe Kejser Christensen, Kobenhavn (DE)

Correspondence Address: BIRCH STEWART KOLASCH & BIRCH, PO BOX 747, FALLS CHURCH, VA, 22040-0747, US

(21) Appl. No.: 12/743,548

(22) PCT Filed: Nov. 26, 2008
(86) PCT No.: PCT/EP08/66223

§ 371 Date: Jul. 9, 2010

Related U.S. Application Data
(60) Provisional application No. 60/990,975, filed on Nov. 29, 2007.

(30) Foreign Application Priority Data
Nov. 28, 2007 (DK) PA200701689

(51) Int. Cl. A61K 31/4245; C07D 271/07; C07D 257/04; A61K 31/41; A61P 25/00; A61P 25/08; A61P 25/28; A61P 25/18; A61P 25/24; A61P 17/00; A61P 9/10; A61P 29/00; A61P 35/00; A61P 1/00

(52) U.S. Cl. 514/364; 548/132; 548/253; 514/381

(57) ABSTRACT

This invention relates to novel phenyl-acetamide and phenyl-propionamide derivatives that are found to be potent modulators of potassium channels and, as such, are valuable candidates for the treatment of diseases or disorders as diverse as those which are responsive to the modulation of potassium channels.

### TECHNICAL FIELD

This invention relates to novel phenyl-acetamide and phenyl-propionamide derivatives that are found to be potent modulators of potassium channels and, as such, are valuable candidates for the treatment of diseases or disorders as diverse as those which are responsive to the modulation of potassium channels.

### BACKGROUND ART

Ion channels are cellular proteins that regulate the flow of ions through cellular membranes of all cells and are classified by their selective permeability to the different of ions (potassium, chloride, sodium etc.). Potassium channels, which represent the largest and most diverse sub-group of ion channels, selectively pass potassium ions and, doing so, they principally regulate the resting membrane potential of the cell and/or modulate their level of excitation.

Dysfunction of potassium channels, as well as other ion channels, generates loss of cellular control resulting in altered physiological functioning and disease conditions. Ion channel blockers and openers, by their ability to modulate ion channel function and/or regain ion channel activity in acquired or inherited channelopathies, are being used in the pharmacological treatment of a wide range of pathological diseases and have the potential to address an even wider variety of therapeutic indications. For instance, the primary indications for potassium channel openers encompass conditions as diverse as diabetes, arterial hypertension, cardiovascular diseases, urinary incontinence, atrial fibrillation, epilepsy, pain, and cancer.

Among the large number of potassium channel types, the large-conductance calcium-activated potassium channel subtype is an obvious site for pharmacological intervention and for the development of new potassium channel modulators. Their physiological role has been especially studied in the nervous system, where they are key regulators of neuronal excitability and of neurotransmitter release, and in smooth muscle, where they are crucial in modulating the tone of vascular, broncho-tracheal, urethral, uterine or gastro-intestinal musculature.

Given these implications, small agents with BK-opening properties could have a potentially powerful influence in the modulation and control of numerous consequences of muscular and neuronal hyperexcitability, such as asthma, urinary incontinence and bladder spasm, gastroenteric hypermotility, psychoses, post-stroke neuroprotection, convulsions, epilepsy, anxiety and pain. As far as the cardiovascular system is concerned, the physiological function of these ion channels represents a fundamental steady state mechanism, modulating vessel depolarisation, vasoconstriction and increases of intravascular pressure, and the development of selective activators of BK channels is seen as a potential pharmacotherapy of vascular diseases, including hypertension, erectile dysfunction, coronary diseases and vascular complications associated with diabetes or hypercholesterolemia.

WO 2006052555 describes niacin receptor antagonists incl. 3-Naphthalen-2-yl-N-[2-(1H-tetrazol-5-yl)-phenyl]-propionamide and 3-(7-Hydroxy-naphthalen-2-yl)-N-[2-(1H-tetrazol-5-yl)-phenyl]-propionamide, and WO 2007044724 describes inhibitors of PIN-1 and/or PIM-3 incl. N45-Chloro-2-(1H-tetrazol-5-yl)-phenyl]-3-phenyl-propionamide and 2-Phenyl-cyclopropanecarboxylic acid [5-chloro-2-(1H-tetrazol-5-yl-phenyl]-amide. However, the phenyl-acetamide and phenyl-propionamide derivatives of the present invention have not been described and their activity as potassium channel modulators certainly not suggested.

### SUMMARY OF THE INVENTION

Is an object of the invention to provide novel phenyl-acetamide and phenyl-propionamide derivatives useful as potassium channel modulators. The phenyl-acetamide and phenyl-propionamide derivatives of the invention may be characterised by Formula I

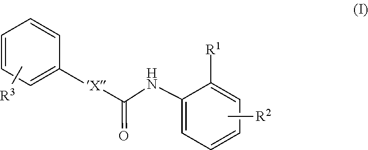

a stereoisomer thereof or a mixture of its stereoisomers, or a pharmaceutically acceptable salt thereof, wherein
′X″ represents a linking group selected from CH₂CH₂, ′OCH₂″, cyclopropyl and cyclopropenyl;
R¹ represents a heteroaryl group selected from 5-oxo-4,5-dihydro-[1,2,4]oxadiazolyl and tetrazolyl; and
R² and R³, independently of each other, represent hydrogen, halo, trifluoromethyl, trifluoromethoxy, nitro and phenyl.

In another aspect the invention provides pharmaceutical compositions comprising a therapeutically effective amount of a phenyl-acetamide and phenyl-propionamide derivative of the invention.

In a third aspect the invention relates to the use of the phenyl-acetamide and phenyl-propionamide derivatives of the invention for the manufacture of pharmaceutical compositions.

In a further aspect the invention provides a method of treatment, prevention or alleviation of a disease or a disorder or a condition of a living animal body, including a human, which disorder, disease or condition is responsive to modulation of potassium channels, which method comprises the step of administering to such a living animal body in need thereof, a therapeutically effective amount of the phenyl-acetamide and phenyl-propionamide derivative of the invention.

Other objects of the invention will be apparent to the person skilled in the art from the following detailed description and examples.

### DETAILED DISCLOSURE OF THE INVENTION

In its first aspect the invention provides novel phenyl-acetamide and phenyl-propionamide derivatives of Formula I

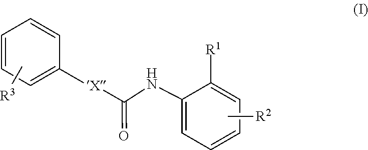

a stereoisomer thereof or a mixture of its stereoisomers, or a pharmaceutically acceptable salt thereof, wherein
′X″ represents a linking group selected from CH₂CH₂, ′OCH₂″, cyclopropyl and cyclopropenyl;
R^{l} represents a heteroaryl group selected from 5-oxo-4,5-dihydro-[1,2,4]oxadiazolyl and tetrazolyl; and
R² and R³, independently of each other, represent hydrogen, halo, trifluoromethyl, trifluoromethoxy, nitro and phenyl; provided, however, if
R¹ represents tetrazolyl; then
R³ independently is not hydrogen.

In a preferred embodiment the phenyl-acetamide and phenyl-propionamide derivative of the invention is a compound of Formula I, a stereoisomer thereof or a mixture of its stereoisomers, or a pharmaceutically acceptable salt thereof, wherein
′X″ represents a linking group selected from CH₂CH₂, ′OCH″₂, cyclopropyl, cyclopropenyl and methyl-cyclopropenyl.

In a more preferred embodiment ′X″ represents CH₂CH₂.

In another more preferred embodiment ′X″ represents ′OCH₂″ (i.e. read in the direction indicated).

In a third more preferred embodiment ′X″ represents a cyclopropyl group.

In a fourth more preferred embodiment ′X″ represents a cyclopropenyl group, in particular a cycloprop-1-ene-1,3-diyl.

In a fifth more preferred embodiment ′X″ represents a methyl-cyclopropenyl group, in particular a 2-methyl-cycloprop-1-ene-1,3-diyl.

In another preferred embodiment the phenyl-acetamide and phenyl-propionamide derivative of the invention is a compound of Formula I, a stereoisomer thereof or a mixture of its stereoisomers, or a pharmaceutically acceptable salt thereof, wherein
R² and R³, independently of each other, represent hydrogen, halo, trifluoromethyl, trifluoromethoxy, nitro and phenyl.

In a more preferred embodiment R² and R³, independently of each other, represent hydrogen, halo, and in particular chloro, or trifluoromethyl.

In another more preferred embodiment R² and R³, independently of each other, represent hydrogen or halo, and in particular chloro.

In a third more preferred embodiment R² represents halo, and in particular chloro; and R³ represents hydrogen or halo, and in particular chloro.

In a fourth more preferred embodiment R² represents halo, and in particular chloro; and R³ represents hydrogen.

In a fifth more preferred embodiment R² represents halo, and in particular chloro; and R³ represents halo, and in particular chloro.

In a most preferred embodiment the phenyl-acetamide and phenyl-propionamide derivative of the invention is
N45-Chloro-2-(5-oxo-4,5-dihydro-[1,2,4]oxadiazol-3-yl)-phenyl]-3-(3-chloro-phenyl)-propionamide;
N45-Chloro-2-(5-oxo-4,5-dihydro-[1,2,4]oxadiazol-3-yl)-phenyl]-3-(4-chloro-phenyl)-propionamide;
2-(3-Chloro-phenoxy)-N-[5-chloro-2-(1H-tetrazol-5-yl)-phenyl]-acetamide;
3-(4-Chloro-phenyl)-N-[5-chloro-2-(1H-tetrazol-5-yl)-phenyl]-propionamide;
3-(3-Chloro-phenyl)-N-[5-chloro-2-(1H-tetrazol-5-yl)-phenyl]-propionamide;
2-Methyl-3-phenyl-cycloprop-2-enecarboxylic acid [5-chloro-2-(5-oxo-4,5-dihydro-[1,2,4]oxadiazol-3-yl)-phenyl]amide;
2-Methyl-3-phenyl-cycloprop-2-enecarboxylic acid [5-chloro-2-(1H-tetrazol-5-yl)-phenyl]-amide;
N-[5-Chloro-2-(5-oxo-4,5-dihydro-[1,2,4]oxadiazol-3-yl)-phenyl]-2-(3-chloro-phenoxy)-acetamide;
2-(4-Chloro-phenyl)-cyclopropanecarboxylic acid [5-chloro-2-(5-oxo-4,5-dihydro-[1,2,4]oxadiazol-3-yl)-phenylFamide; or
2-(4-Chloro-phenyl)-cyclopropanecarboxylic acid [5-chloro-2-(1H-tetrazol-5-yl)-phenyl]-amide;
   a stereoisomer thereof or a mixture of its stereoisomers, or a pharmaceutically acceptable salt thereof.

Any combination of two or more of the embodiments described herein is considered within the scope of the present invention.

### DEFINITION OF SUBSTITUENTS

In the context of this invention halo represents fluoro, chloro, bromo or iodo.

### Pharmaceutically Acceptable Salts

The phenyl-acetamide and phenyl-propionamide derivatives of the invention may be provided in any form suitable for the intended administration. Suitable forms include pharmaceutically (i.e. physiologically) acceptable salts, and pre- or prodrug forms of the phenyl-acetamide and phenyl-propionamide derivative of the invention.

Examples of pharmaceutically acceptable salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a phenyl-acetamide and phenyl-propionamide derivative of the invention include, without limitation, the sodium, the potassium, the calcium, the magnesium, the lithium, and the ammonium salt, and the like, of a phenyl-acetamide and phenyl-propionamide derivative of the invention containing an anionic group. Such cationic salts may be formed by procedures well known and described in the art.

### Steric Isomers

It will be appreciated by those skilled in the art that the compounds of the present invention may exist in different stereoisomeric forms, including enantiomers, diastereomers, as well as geometric isomers (cis-trans isomers). The invention includes all such isomers and any mixtures thereof including racemic mixtures.

Racemic forms can be resolved into the optical antipodes by known methods and techniques. One way of resolving racemates into the optical antipodes is based upon chromatography on an optical active matrix. Racemic compounds of the present invention can thus be resolved into their optical antipodes, e.g., by fractional crystallisation of D- or L- (tartrates, mandelates, or camphorsulphonate) salts for example.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optically active starting materials or intermediates.

### Methods of Preparation

The compounds according to the invention may be prepared by conventional methods for chemical synthesis, e.g. those described in the working examples.

### Biological Activity

The phenyl-acetamide and phenyl-propionamide derivatives of the invention have been found to possess potassium channel modulating activity as measured by standard electrophysiological methods. Due to their activity at the potassium channels, the phenyl-acetamide and phenyl-propionamide derivatives of the invention are considered useful for the treatment of a wide range of diseases and conditions.

In a special embodiment, the phenyl-acetamide and phenyl-propionamide derivatives of the invention are considered useful for the treatment, prevention or alleviation of a respiratory disease, epilepsy, partial epilepsy, convulsions, seizures, absence seizures, vascular spasms, coronary artery spasms, motor neuron diseases, myokymia, renal disorders, polycystic kidney disease, bladder hyperexcitability, bladder spasms, urinogenital disorders, urinary incontinence, bladder outflow obstruction, erectile dysfunction, gastrointestinal dysfunction, gastrointestinal hypomotility disorders, gastrointestinal motility insufficiency, postoperative ileus, constipation, gastroesophageal reflux disorder, secretory diarrhoea, an obstructive or inflammatory airway disease, ischaemia, cerebral ischaemia, ischaemic heart disease, angina pectoris, coronary heart disease, ataxia, traumatic brain injury, stroke, Parkinson's disease, bipolar disorder, psychosis, schizophrenia, autism, anxiety, mood disorders, depression, manic depression, psychotic disorders, dementia, learning deficiencies, age related memory loss, memory and attention deficits, Alzheimer's disease, amyotrophic lateral sclerosis (ALS), dysmenorrhoea, narcolepsy, sleeping disorders, sleep apnoea, Reynaud's disease, intermittent claudication, Sjögren's syndrome, xerostomia, cardiovascular disorders, hypertension, myotonic dystrophy, myotonic muscle dystrophia, spasticity, xerostomia, diabetes Type II, hyperinsulinemia, premature labour, cancer, brain tumours, inflammatory bowel disease, irritable bowel syndrome, colitis, colitis Crohn, immune suppression, hearing loss, migraine, pain, neuropathic pain, inflammatory pain, trigeminal neuralgia, vision loss, rhinorrhoea, ocular hypertension (glaucoma) or baldness.

In a more preferred embodiment, the phenyl-acetamide and phenyl-propionamide derivatives of the invention are considered useful for the treatment, prevention or alleviation of a respiratory disease, urinary incontinence, erectile dysfunction, anxiety, epilepsy, psychosis, schizophrenia, bipolar disorder, depression, amyotrophic lateral sclerosis (ALS), Parkinson's disease or pain.

In another more preferred embodiment, the phenyl-acetamide and phenyl-propionamide derivatives of the invention are considered useful for the treatment, prevention or alleviation of psychosis, schizophrenia, bipolar disorder, depression, epilepsy, Parkinson's disease or pain.

In a third more preferred embodiment, the phenyl-acetamide and phenyl-propionamide derivatives of the invention are considered useful for the treatment, prevention or alleviation of pain, mild or moderate or severe pain, pain of acute, chronic or recurrent character, pain caused by migraine, postoperative pain, phantom limb pain, inflammatory pain, neuropathic pain, chronic headache, central pain, pain related to diabetic neuropathy, to post therapeutic neuralgia, or to peripheral nerve injury.

In a fourth more preferred embodiment, the phenyl-acetamide and phenyl-propionamide derivatives of the invention are considered useful for the treatment, prevention or alleviation of cardiac ischemia, ischemic heart disease, hypertrophic heart, cardiomyopathy or failing heart.

In a fifth more preferred embodiment, the compounds of the invention are considered useful for the treatment, prevention or alleviation of a cardiovascular disease. In a more preferred embodiment the cardiovascular disease is atherosclerosis, ischemia/reperfusion, hypertension, restenosis, arterial inflammation, myocardial ischaemia or ischaemic heart disease.

In an sixth more preferred embodiment, the compounds of the invention are considered useful for obtaining preconditioning of the heart. Preconditioning, which includes ischemic preconditioning and myocardial preconditioning, describes short periods of ischemic events before initiation of a long lasting ischemia. The compounds of the invention are believed having an effect similar to preconditioning obtained by such ischemic events. Preconditioning protects against later tissue damage resulting from the long lasting ischemic events.

In a seventh more preferred embodiment, the phenyl-acetamide and phenyl-propionamide derivatives of the invention are considered useful for the treatment, prevention or alleviation of schizophrenia, depression or Parkinson's disease.

In an eighth more preferred embodiment, the compounds of the invention are considered useful for the treatment, prevention or alleviation of an obstructive or inflammatory airway disease. In a more preferred embodiment the obstructive or inflammatory airway disease is respiratory failure, adult respiratory distress syndrome, asthma, nocturnal asthma, exercise induced bronchospasm, chronic obstructive pulmonary disease, giant bullae, acute bronchitis, chronic bronchitis, emphysema, reversible obstructive airway disease, bronchiectasis, bronchiolitis, cystic fibrosis, eatelectasis, pulmonary embolism, pneumonia, gastroesophageal reflux disease (GERD), lung abscess, hypersensitivity of the lung, hypersensitivity pneumonitis, eosinophilic pneumonias, allergic bronchopulmonary aspergillosis, or Goodpasture's syndrome. In an even more preferred embodiment the obstructive or inflammatory airway disease is an airway hyperreactivity, a pneumoconiosis such as aluminosis, anthracosis, asbestosis, chalicosis, ptilosis, siderosis, silicosis, tabacosis and byssinosis, a chronic obstructive pulmonary disease (COPD), bronchitis, excerbation of airways hyperreactivity or cystic fibrosis.

In its most preferred embodiment the obstructive airway disease is chronic obstructive pulmonary disease (COPD).

In a ninth more preferred embodiment the compound of the invention is used in a combination with conventional bronchodilators, in particular the beta(2)-adrenoceptor agonists. Examples of bronchodilator drugs for use according to the invention include salbutamol (Albuterol, Ventolin) and formoterol (Foradil).

In a tenth more preferred embodiment the phenyl-acetamide and phenyl-propionamide derivatives of the invention are considered useful for the treatment, prevention or alleviation of a sexual dysfunction, incl. male sexual dysfunction and female sexual dysfunction, and incl. male erectile dysfunction.

In an even more preferred embodiment the phenyl-acetamide and phenyl-propionamide derivatives of the invention may be co-administered with a phosphodiesterase inhibitor, in particular a phosphodiesterase 5 (PDE5) inhibitor, e.g. sildenafil, tadalafil, vardenafil and dipyridamole, or with an agent that potentiates endothelium-derived hyperpolarizing factor-mediated responses, in particular calcium dobesilate or similar 2,5-dihydroxybenzenesulfonate analogs.

In a most preferred embodiment the phenyl-acetamide and phenyl-propionamide derivatives of the invention is used in a combination therapy together with sildenafil, tadalafil, vardenafil or calcium dobesilate.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

Preferred phenyl-acetamide and phenyl-propionamide derivatives of the invention show a biological activity in the sub-micromolar and micromolar range, i.e. of from below 1 to about 100 μM.

### Pharmaceutical Compositions

In another aspect the invention provides novel pharmaceutical compositions comprising a therapeutically effective amount of a phenyl-acetamide and phenyl-propionamide derivative of the invention.

While a phenyl-acetamide and phenyl-propionamide derivative of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the phenyl-acetamide and phenyl-propionamide derivative of the invention together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in drage, in powder, or in liquid form, and parenteral administration, in particular cutaneous, subcutaneous, intramuscular, or intravenous injection. The pharmaceutical composition of the invention can be manufactured by any person skilled in the art, by use of standard methods and conventional techniques, appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

In another aspect the invention provides a method of treatment, prevention or alleviation of a disease, disorder or condition of a living animal body, including a human, which disorder, disease or condition is responsive to activation of a potassium channel, which method comprises the step of administering to such a living animal body in need thereof, a therapeutically effective amount a compound capable of activating the potassium channel, or a pharmaceutically acceptable salt thereof.

The preferred medical indications contemplated according to the invention are those stated above.

It is at present contemplated that a suitable dosage of the active pharmaceutical ingredient (API) is within the range of from about 0.1 to about 1000 mg API per day, more preferred of from about 1 to about 500 mg API per day, most preferred of from about 1 to about 100 mg API per day, dependent, however, upon the exact mode of administration, the form in which it is administered, the indication considered, the subject and in particular the body weight of the subject involved, and further the preference and experience of the physician or veterinarian in charge.

### BRIEF DESCRIPTION OF THE DRAWINGS

The present invention is further illustrated by reference to the accompanying drawing, in which FIGS. 1A and 1B show the effect of Compound 8 (i.e. N-[5-Chloro-2-(5-oxo-4,5-dihydrop-[1,2,4]oxadiazol-3-yl)-phenyl]-2-(3-chloro-phenoxy)-acetamide) on the voltage dependence of BK_{ca} channels expressed in Xenopus Oocytes:

FIG. 1A shows conductance (μS) vs. membrane potential (mV) in the absence (Control) of Compound 8 and in the presence of 0.01 to 31.6 μM of Compound 8; and

FIG. 1B shows the concentration-response relationship for the left-shift of the BK_{ca}-activation curve induced by Compound 8; i.e. ΔV (mV) vs. log [c] (M). The calculated EC₅₀-value is 8.2 μM and the maximal left-shift for the BK-activation curve is −94 mV.

### EXAMPLES

The invention is further illustrated with reference to the following examples, which are not intended to be in any way limiting to the scope of the invention as claimed.

### Example 1

### Preparatory Example

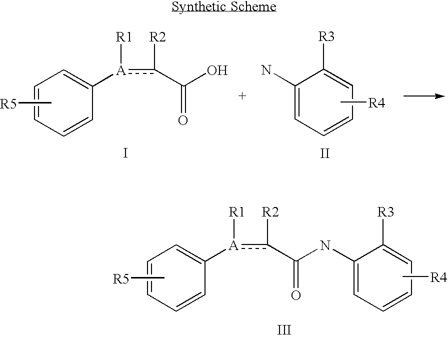

### Intermediates I

3-(3-Chloro-phenyl)-propionic acid (A)
3-(4-Chloro-phenyl)-propionic acid (B)
(3-Chloro-phenoxy)-acetic acid (C)
2-Methyl-3-phenyl-cycloprop-2-enecarboxylic acid (D)

Intermediates A, B, C and D are commercially-available.

When Intermediate I is represented by a suitably-substituted phenyl-cyclopropanecarboxylic acid it is easily prepared by conventional methods described in the literature. Both cis and trans isomers are possible and distinguishable by their NMR spectra, upon consideration of the constants of the vicinal protons. (Trans)-phenylcycloprpanecarboxylic acids were prepared as described by Pryde et al. [Pryde D C, Cook A S, Burring D J, Jones L H, Foll S, Platts M Y, Sanderson V, Corless M, Stobie A, Middleton D S, Foster L, Barker L, Van der Graaf P, Stacey P, Kohl C, Coggon S, Beaumont K: “Novel selective inhibitors of neutral endopeptidase for the treatment of female sexual arousal disorder”; Bioorganic & Medicinal Chemistry 2007 15 (1) 142-159], whereas (cis)-phenylcycloprpanecarboxylic acids were prepared as described by Diaz-Requejo et al. [Diaz-Requejo M M, Caballero A, Belderrain T R, Nicasio M C, Trofimenko S & Perez P J: in “Copper(I)-Homoscorpionate Catalysts for the Preferential, Kinetically Controlled C is Cyclopropanation of r-Olefins with Ethyl Diazoacetate”; J. Am. Chem. Soc. 2002 124 (6) 978].

As an example, the preparation of (cis)-2-(4-Chloro-phenyl)-cyclopropanecarboxylic acid (E) is reported.
2-(4-Chloro-phenyl)-cyclopropanecarboxylic acid (E)

Intermediate E was synthesised as outlined and described below.

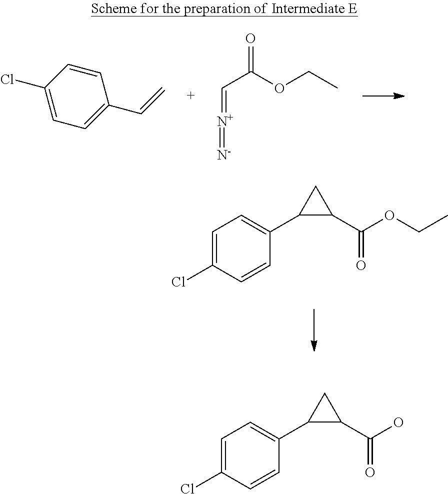

Copper (I) iodide (0.042 g, 0.025 eq) and hydrotris-(3-phenylpyrazol-1-yl)borate potassium salt (0.105 g, 0.025 eq) were suspended in dry dichloromethane (96 ml) and the flask was evacuated and back filled with nitrogen. The suspension was stirred at room temperature for 2 hours, then 4-chlorostyrene (6.07 g, 5 eq) was added via a syringe, followed by the addition of ethyldiazoacetate (1 g, 1 eq). Stirring was continued at room temperature overnight and the reaction was then quenched with 1M HCl (100 ml). Stirring continued for additional 10 min and the two phases were then separated. The aqueous phase was extracted with additional dichloromethane and the combined organic phases were dried over MgSO₄, filtered and evaporated to dryness, to afford 8 g of crude 2-(4-chloro-phenyl)-cyclopropanecarboxylic acid ethyl ester. This latter was dissolved in methanol (50 ml) and 4M NaOH added (50 ml). The reaction mixture was refluxed for 2 hours, cooled to room temperature, diluted with water (100 ml) and extracted with diethyl ether. The aqueous phase was acidified and finally extracted with dichloromethane, to afford 1.50 g of crude 2-(4-chloro-phenyl)-cyclopropanecarboxylic acid. This was purified by flash chromatography eluting with 10% ethyl acetate in hexane (0.700 g, yield 41%).

### Intermediates II

3-(2-Amino-4-chloro-phenyl)-4H-(1,2,4]oxadiazol-5-one (A)
5-Chloro-2-(1H-tetrazol-5-yl)-phenylamine (B)

Intermediates A and B were prepared as described by Valgeirsson et al. in Journal of Medicinal Chemistry 2004 47 (27) 6948-6957.

Final compounds: Ill

### General Procedure

To a suspension of Intermediate I (A-E) (1 eq) in dichloromethane (DCM), 1-ethyl-3-(3-dimethylaminopropyl) carbodiimide HCl (EDC.HCl) (2 eq) and 4-(dimethylamino)pyridine (DMAP) (3 eq) are added. The resulting mixture is stirred for 10 min and Intermediate II (A-B) (1 eq) is added. The solution is stirred at room temperature overnight, diluted with DCM, washed with 1.5 N HCl and water, dried and evaporated to dryness, to afford the title compound as a crude solid, which is purified either by crystallisation (CR) (AcOEt or DMSO/water) or flash chromatography (FC) (20-90% yield).

| Intermediate I | Intermediate II | Final compound | Purification |
|---|---|---|---|
| IA | IIA | 1 | CR |
| IB | IIA | 2 | CR |
| IC | IIB | 3 | CR |
| IB | IIB | 4 | CR |
| IA | IIB | 5 | CR |
| ID | IIA | 6 | CR |
| ID | IIB | 7 | CR |
| IC | IIA | 8 | CR |
| IE | IIA | 9 | FC |
| IE | IIB | 10 | FC |

N-[5-Chloro-2-(5-oxo-4,5-dihydro-(1,2,4]oxadiazol-3-yl)-phenyl]-3-(3-chloro-phenyl)-propionamide (1)

LC-ESI−HRMS of [M−H]- shows 376.024 Da. Calc. 376.025573 Da, dev. -4.2 ppm.
N-[5-Chloro-2-(5-oxo-4,5-dihydro-[1,2,4]oxadiazol-3-yl)-phenyl]-3-(4-chloro-phenyl)-propionamide (2)

LC-ESI−HRMS of [M−H]- shows 376.0261 Da. Calc. 376.025573 Da, dev. 1.4 ppm.
2-(3-Chloro-phenoxy)-N-[5-chloro-2-(1H-tetrazol-5-yl)-phenyl]-acetamide (3)

LC-ESI−HRMS of [M−H]- shows 362.0193 Da. Calc. 362.021156 Da, dev.-5.1 ppm.
3-(4-Chloro-phenyl)-N-[5-chloro-2-(1H-tetrazol-5-yl)-phenyl]-propionamide (4)

LC-ESI−HRMS of [M−H]- shows 360.041 Da. Calc. 360.041891 Da, dev. -2.5 ppm.
3-(3-Chloro-phenyl)-N45-chloro-2-(1H-tetrazol-5-v1)-phenyl]-propionamide (5)

LC-ESI−HRMS of [M+H]+shows 362.0587 Da. Calc. 362.057541 Da, dev. 3.2 ppm.
2-Methyl-3-phenyl-cycloprop-2-enecarboxylic acid [5-chloro-2-(5-oxo-4,5-dihydro-[1,2,4]oxadiazol-3-yl)-phenyl]-amide (6)

LC-ESI−HRMS of [M+H]+shows 368.0818 Da. Calc. 368.080195 Da, dev. 4.4 ppm.
2-Methyl-3-phenyl-cycloprop-2-enecarboxylic acid [5-chloro-2-(1H-tetrazol-5-yl)-phenyl]-amide (7)

LC-ESI−HRMS of [M+H]+shows 352.0964 Da. Calc. 352.096513 Da, dev.-0.3 ppm.
N15-Chloro-2-(5-oxo-4,5-dihydro-[1,2,4]oxadiazol-3-yl)-phenyl]-2-(3-chloro-phenoxy)-acetamide (8)

LC-ESI−HRMS of [M+H]+shows 380.0223 Da. Calc. 380.020488 Da, dev. 4.8 ppm.
2-(4-Chloro-phenyl)-cyclopropanecarboxylic acid[5-chloro-2-(5-oxo-4,5-dihydro-[1,2,4]oxadiazol-3-yl-phenyl]-amide (9)

LC-ESI−HRMS of [M+H]+shows 390.0431 Da. Calc. 390.041223 Da, dev. 4.8 ppm. M.p. 162.9-170.8° C.
2-(4-Chloro-phenyl)-cyclopropanecarboxylic acid[5-chloro-2-(1H-tetrazol-5-yl)-phenyl]-amide (10)

LC-ESI−HRMS of [M+H]+shows 374.0568 Da. Calc. 374.057541 Da, dev.-2 ppm. M.p. 242.5-243.8° C.

### Example 2

### Biolocical Activity

In this example the BK channel opening activity of Compound 8 is determined using BK channels heterologously expressed in Xenopus iaevis oocytes.

The electrical current through the BK channel was measured using conventional two-electrode voltage clamp. BK currents were activated by repeating ramp protocols. In brief, the membrane potential was continuously changed from −120 mV to +120 mV within a 2 s period. The threshold for BK activation is approximately +30 mV under control conditions. Compounds were applied for 100 s during which the ramp protocol was repeated 10 times with 10 s intervals. In between the ramp protocols the membrane potential was clamped at −80 mV. The first three compound applications were control blanks where the current level is allowed to stabilize. During the subsequent 8 applications increasing concentrations (0.01-31.6 μM) of compound was applied and a marked increase in the current level at depolarizing potentials was observed.

In order to evaluate the ability of the compounds to shift the BK activation curve towards lower membrane potentials, the BK current was transformed into conductance by using Ohm's law g=I/(E_{memb}−Eᵣₑᵥ), where g is the conductance, I is the current, E_{memb} is the membrane potential and Eᵣₑᵥ is the reversal potential. The extracellular solution for these experiments contained 2.5 mM K+ and the intracellular K⁺ concentration of an oocyte was estimated to be 100 mM. Under those conditions, Nernst equation predicts a reversal potential of Eᵣₑᵥ=−93.2 mV. The control conductance level at a membrane potential of +100 mV was calculated, and the compound effect was evaluated as the potential difference, ΔW, to the membrane potential at which the same conductance level was obtained in the presence of compound.

The concentration response curve for this potential difference was fitted to the sigmoidal logistic equation: ΔV=ΔVₘₐₓ/(1+(EC₅₀/[compound])ⁿ), where ΔV. represents the maximal left shift of the BK activation curve, EC₅₀ is the concentration causing a half maximal response, and n is the slope coefficient.

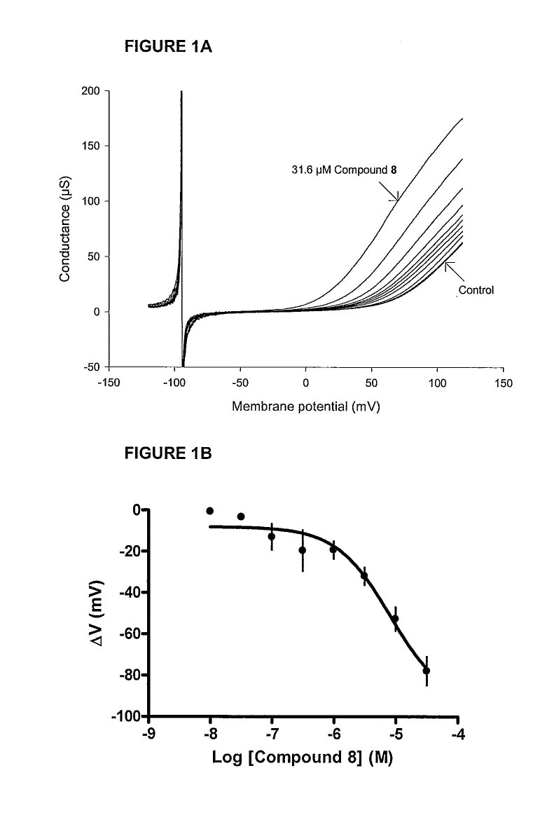

The results of this determination are presented in FIGS. 1A and 1B. The calculated EC₅₀ and ΔVmax values for Compound 8 were 8.2 μM and -94 mV, respectively.

1-9. (canceled)

10. A phenyl-acetamide and phenyl-propionamide derivative of Formula I 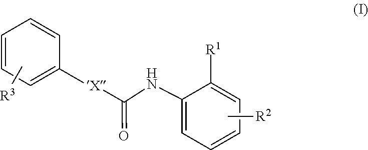
a stereoisomer thereof or a mixture of its stereoisomers, or a pharmaceutically acceptable salt thereof, wherein
′X″ represents a linking group selected from CH₂CH₂, ′OCH₂″, cyclopropyl and cyclopropenyl;
R¹ represents a heteroaryl group selected from 5-oxo-4,5-dihydro-[1,2,4]oxadiazolyl and tetrazolyl; and
R² and R³, independently of each other, represent hydrogen, halo, trifluoromethyl, trifluoromethoxy, nitro and phenyl.

11. The phenyl-acetamide and phenyl-propionamide derivative of claim 10, a stereoisomer thereof or a mixture of its stereoisomers, or a pharmaceutically acceptable salt thereof, wherein
′X″ represents a linking group selected from CH₂CH₂, ′OCH₂″, cyclopropyl, cyclopropenyl and methyl-cyclopropenyl.

12. The phenyl-acetamide and phenyl-propionamide derivative of claim 10, a stereoisomer thereof or a mixture of its stereoisomers, or a pharmaceutically acceptable salt thereof, wherein
R² and R³, independently of each other, represent hydrogen, halo, trifluoromethyl, trifluoromethoxy, nitro and phenyl.

13. The phenyl-acetamide and phenyl-propionamide derivative of claim 10, which is
N-[5-Chloro-2-(5-oxo-4,5-dihydro-[1,2,4]oxadiazol-3-yl)-phenyl]-3-(3-chloro-phenyl)-propionamide;
N-[5-Chloro-2-(5-oxo-4,5-dihydro- [1,2,4]oxadiazol-3-yl)-phenyl]-3-(4-chloro-phenyl)-propionamide;
2-(3-Chloro-phenoxy)-N- [5-chloro-2-(1H-tetrazol-5-yl)-phenyl]-acetamide;
3-(4-Chloro-phenyl)-N-[5- chloro-2-(1H-tetrazol-5-yl)-phenyl]-propionamide;
3-(3-Chloro-phenyl)-N-[5- chloro-2-(1H-tetrazol-5-yl)-phenyl]-propionamide;
2-Methyl-3-phenyl- cycloprop-2-enecarboxylic acid [5- chloro-2-(5- oxo-4,5-dihydro-[1,2,4]oxadiazol-3-yl)-phenyl]-amide;
2-Methyl-3-phenyl- cycloprop-2- enecarboxylic acid [5-chloro-2-(1H-tetrazol-5-yl)-phenyl]-amide;
N-[5-Chloro-2-(5-oxo-4,5-dihydro- [1,2,4]oxadiazol-3-yl)-phenyl]-2-(3-chloro-phenoxy)-acetamide;
2-(4-Chloro-phenyl)-cyclopropanecarboxylic acid [5-chloro-2-(5-oxo-4,5-dihydro-[1,2,4]oxadiazol-3-yl)-phenyl]-amide; or
2-(4-Chloro-phenyl)-cyclopropanecarboxylic acid [5-chloro-2-(1H-tetrazol-5-yl)-phenyl]-amide;
a stereoisomer thereof or a mixture of its stereoisomers, or a pharmaceutically acceptable salt thereof.

14. A pharmaceutical composition comprising a therapeutically effective amount of the phenyl-acetamide and phenyl-propionamide derivative of claim 10, a stereoisomer thereof or a mixture of its stereoisomers, or a pharmaceutically acceptable salt thereof, together with one or more adjuvants, excipients, carriers and/or diluents.

15. A method of treatment, prevention or alleviation of a disease or a disorder or a condition of a living animal body, including a human, which disorder, disease or condition is responsive to modulation of potassium channels, which method comprises the step of administering to such a living animal body in need thereof, a therapeutically effective amount of the phenyl-acetamide and phenyl-propionamide derivative according to claim 10, a stereoisomer thereof or a mixture of its stereoisomers, or a pharmaceutically acceptable salt thereof.

16. The method according to claim 15, wherein the disease, disorder or condition is a respiratory disease, epilepsy, convulsions, seizures, absence seizures, vascular spasms, coronary artery spasms, motor neuron diseases, myokymia, renal disorders, polycystic kidney disease, bladder hyperexcitability, bladder spasms, urinogenital disorders, urinary incontinence, bladder outflow obstruction, erectile dysfunction, gastrointestinal dysfunction, gastrointestinal hypomotility disorders, gastrointestinal motility insufficiency, postoperative ileus, constipation, gastroesophageal reflux disorder, secretory diarrhoea, an obstructive or inflammatory airway disease, ischaemia, cerebral ischaemia, ischaemic heart disease, angina pectoris, coronary heart disease, ataxia, traumatic brain injury, stroke, Parkinson's disease, bipolar disorder, psychosis, schizophrenia, autism, anxiety, mood disorders, depression, manic depression, psychotic disorders, dementia, learning deficiencies, age related memory loss, memory and attention deficits, Alzheimer's disease, amyotrophic lateral sclerosis (ALS), dysmenorrhea, narcolepsy, sleeping disorders, sleep apnea, Reynaud's disease, intermittent claudication, Sjögren's syndrome, xerostomia, cardiovascular disorders, hypertension, myotonic dystrophy, myotonic muscle dystrophia, spasticity, xerostomi, diabetes Type II, hyperinsulinemia, premature labour, cancer, brain tumors, inflammatory bowel disease, irritable bowel syndrome, colitis, colitis Crohn, immune suppression, hearing loss, migraine, pain, neuropathic pain, inflammatory pain, trigeminal neuralgia, vision loss, rhinorrhoea, ocular hypertension (glaucoma) or baldness.
